(19) United States
(12) Patent Application Publication
(10) Pub. No.: US 2018/0193660 A1
(43) Pub. Date: Jul. 12, 2018

(54) Intranasal Insert for OFC Neuroprotection

(71) Applicant: Janssen Pharmaceutica NV, Beerse (BE)

(72) Inventors: Thomas M. DiMauro, Southboro, MA (US); Kevin Wildenhaus, Plymouth, MI (US); John Pracyk, Cumberland, RI (US); Michael Luedtke, Bellingham, MA (US)

(73) Assignee: Janssen Pharmaceutica NV, Beerse (BE)

(21) Appl. No.: 15/792,944

(22) Filed: Oct. 25, 2017

Related U.S. Application Data
(60) Provisional application No. 62/445,300, filed on Jan. 12, 2017.

(51) Int. Cl. A61N 5/06

(52) U.S. Cl. CPC A61N 5/0603; A61N 5/0622; A61N 2005/0645; A61N 2005/0607; A61N 2005/0659

(57) ABSTRACT

An aesthetically pleasing intranasal NIR/red light emitting device, wherein the device is a) secured in a first nostril by a spring clip securely contacting the inner surface of the opposite nostril, or b) secured in a first nostril by a retaining element (such as an adhesive) adapted to containing substantially only the inner surface of the first nostril.

### CONTINUITY DATA

This application claims priority from copending provisional application U.S. Ser. No. 62/445,300, entitled “Intranasal Insert for OFC Neuroprotection”, filed Jan. 12, 2017, (DiMauro et al.,) (Docket PRD3443USPSP), the specification of which is incorporated by reference in its entirety.

### BACKGROUND OF THE INVENTION

US Published Patent Application 2014-0358199 (Lim) discloses the intranasal delivery of the infrared light to the orbitofrontal cortex of the brain. The commercial embodiment of this application appears to be the Vielight 810® device. The Vielight 810® device comprises an infrared LED that is inserted into the nose and is powered by a battery pack. A wire connects the LED to the battery pack. The LED device is secured in the nose by virtue of a clip secured to the lateral surface of the nose.

U.S. Pat. No. 8,734,498 (Codman) discloses a hand-held intranasal light device comprising an infrared LED powered by a battery contained within the handle of the device.

Taiwanese Published Patent Application TWM 280228 discloses a device that intranasally delivers red light to the nasal cavity, wherein light is bilaterally delivered to the nasal cavity. The pair of cylindrical emitters are connected by a flat plank whereby the cylinders extend substantially perpendicular to the flat plank.

### SUMMARY OF THE INVENTION

In accordance with the present application, there is provided an intranasal light-emitting device comprising:
i) a proximal portion comprising a connecting cap comprising an LED actuator;
ii) an intermediate portion comprising a tube that houses a power source in its bore and a proximal mating connector, and
iii) a distal portion comprising a near infrared LED,
   wherein the dimensions of the device are such that the device fits snuggly within the nostril and remains in place even under human activity.

In accordance with the present application, there is provided a method comprising:
a) inserting into the patient's nostril a light-emitting device so that at least 70% of the device fits inside the patient's nostril, wherein the device comprises:
   i) a proximal portion comprising a connecting cap comprising an LED actuator;
   ii) an intermediate portion comprising a tube that houses a power source in its bore and a proximal mating connector, and
   iii) a distal portion comprising a near infrared LED, and
b) actuating the device by placing the LED in electrical connection with the power source.

In accordance with the present application, there is provided an intranasal light-emitting device comprising:
i) a proximal portion comprising a connecting cap comprising an LED actuator;
ii) an intermediate portion comprising a tube that houses a power source in its bore and a proximal mating connector, and
iii) a distal portion comprising a near infrared LED,
   wherein the device further includes electronics adapted to provide a substantially constant current to the LED.
   In accordance with the present application, there is provided an intranasal light-emitting device comprising:
i) a proximal portion comprising a connecting cap comprising an LED actuator;
ii) an intermediate portion comprising a tube that houses a power source in its bore and a proximal mating connector, and
iii) a distal portion comprising a near infrared LED,
   wherein the near infrared LED is surrounded by a distal tip that is adapted to emit the NIR light distally.

Accordingly, there is now provided an improved intranasal light delivery device comprising a NIR/red light emitter, wherein the device is secured in a first nostril by a spring clip secured in the opposite nostril.

In other embodiments, there is now provided an improved intranasal light delivery device comprising a NIR/red light emitter, wherein the device is secured in a first nostril by an adhesive that coats the proximal portion of the device. Preferably, the device has a substantially cylindrical shape adapted to fit within the user's nostril, and the coating is provided on the proximal portion of the substantially cylindrical surface. Preferably, the substantially cylindrical shape has an axial cross-section substantially similar to the axial cross-section of the user's nostril.

### DESCRIPTION OF THE FIGURES

FIG. 1 discloses a sagittal cross section of a brain, with the orbitofrontal cortex highlighted as OFC.

FIG. 2 discloses an upper roof of the nasal cavity being a wafer-thin ledge of porous bony tissue located beneath the prefrontal cortex portion of the brain and above the nasal cavity.

FIG. 3 discloses an axial cross-section of the skull revealing the location of the cribriform plate (CP).

FIG. 4 discloses an axial cross-section of the skull revealing the location of the orbitofrontal cortex (OFC) above the eye sockets of the skull.

FIG. 5 discloses another sagittal cross section of a brain, with the orbitofrontal cortex region highlighted as OFC.

FIG. 6A discloses a bilateral light emitting device having a spring clip connecto, wherein the device is in a loaded condition as when it is inserted into a pair of nostrils.

FIG. 6B discloses the bilateral light emitting device of FIG. 6a, wherein the device is in an unloaded condition.

FIG. 6C discloses the angle alpha formed by the tubes of the unloaded device of FIG. 6b.

FIG. 6D discloses a detailed view of a single emitter of the device of FIG. 6a.

FIG. 6E discloses a distal portion of a single emitter of the device of FIG. 6a.

FIG. 7A discloses a unilateral light emitting device having a spring clip retainer, wherein the device is in a loaded condition as when it is inserted into a pair of nostrils.

FIG. 7B discloses the unilateral light emitting device of FIG. 7a, wherein the device is in an unloaded condition.

FIG. 8 discloses a unilateral light emitting device having an adhesive layer disposed about its tube.

FIG. 9 discloses an embodiment of the intranasal device.

FIG. 10 shows an axial cross section of a nasal cavity.

FIGS. 11A-C show various views of a preferred intranasal device.

### DETAILED DESCRIPTION OF THE INVENTION

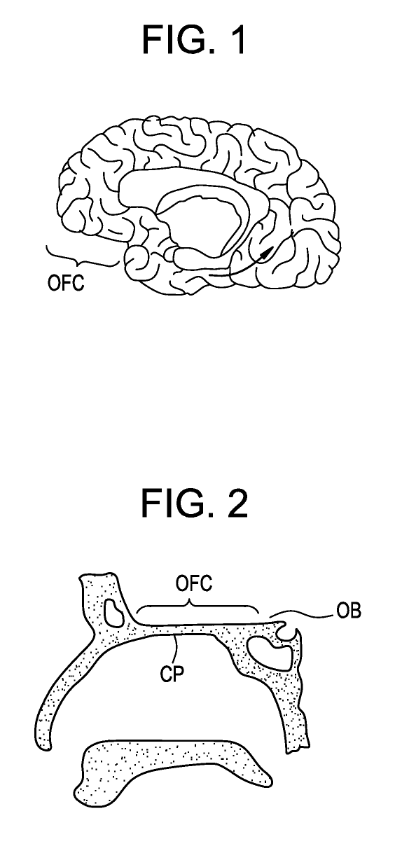

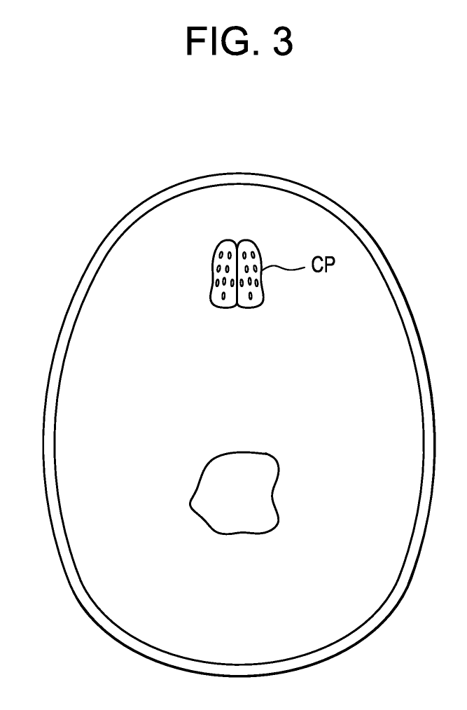

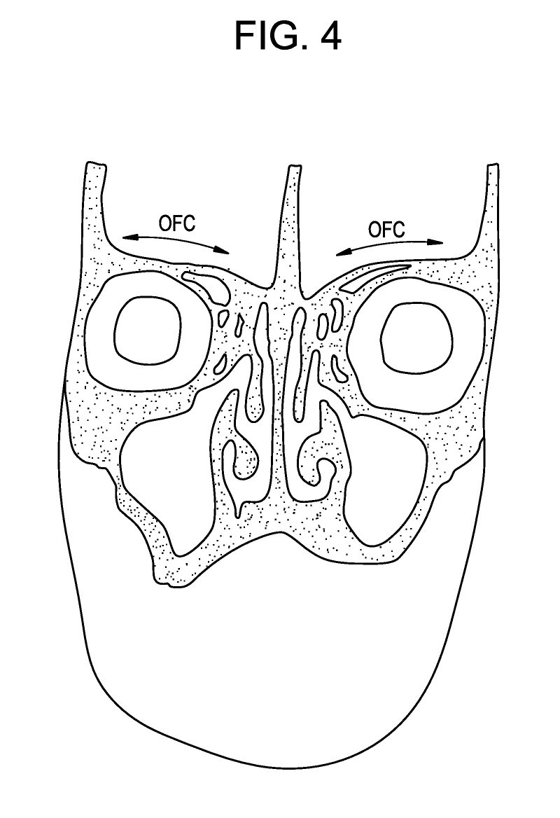

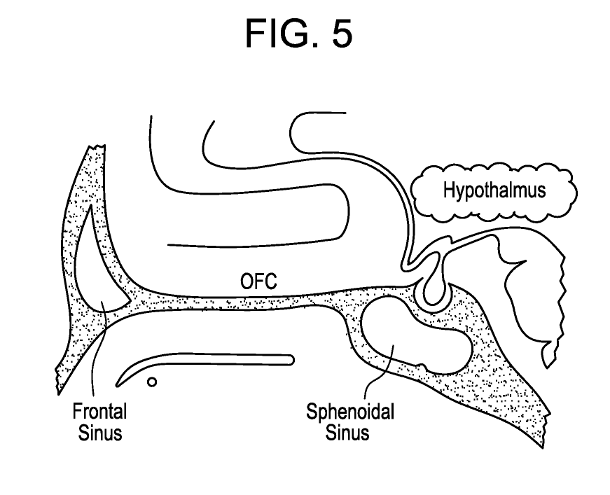

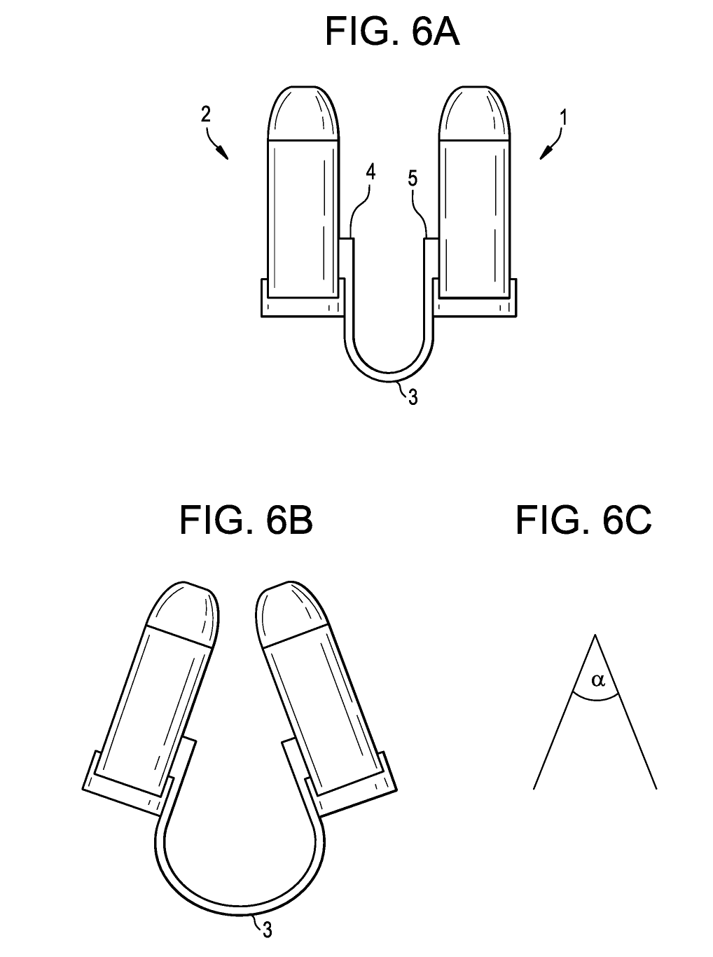

Now referring to FIG. 1, there is provided a sagittal cross section of a brain, with the orbitofrontal cortex region highlighted as OFC. Now referring to FIG. 2, the upper roof of the nasal cavity is a wafer-thin ledge of porous bony tissue located beneath both the orbitofrontal cortex portion of the brain (OFC) and the olfactory bulb (OB), and above the nasal cavity. This wafer-thin ledge of porous bony tissue holds the cribriform plate (CP). Now referring to FIG. 3, there is provided an axial cross-section of the skull revealing the location of the cribriform plate (CP). Now referring to FIG. 4, there is provided an axial cross-section of the skull revealing the location of the orbitofrontal cortex (OFC) above the eye sockets of the skull. Now referring to FIG. 5, there is provided another sagittal cross section of a brain, with the orbitofrontal cortex region highlighted as OFC.

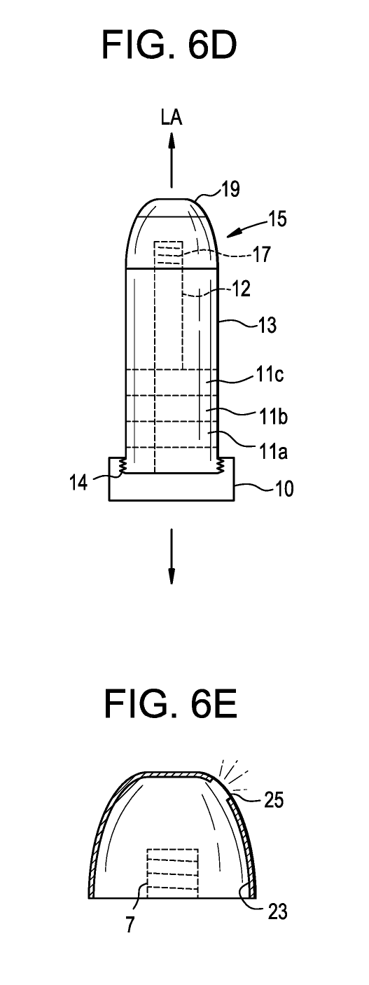

Now referring to FIGS. 6A-E, in some embodiments, the device has bilateral infrared/red light emitters. Preferably, in this embodiment, the device comprises:

a) first and second emitters 1 and 2, each emitter having:
i) a proximal portion comprising an LED actuator (such as a threaded on/off knob 10);
ii) an intermediate portion comprising first and second substantially cylindrical tubes 13 that house respective batteries 11a-c in their bores and a proximal mating thread 14, and
iii) a distal portion 15 comprising an LED 17 having leads 12, wherein a first lead contacts the distal pole of the distalmost battery 11c and a second lead contacts the proximal pole of the proximalmost battery 11a. and
   b) a springy curved connector 3 having opposite ends 4 and 5,
   wherein the tube of each emitter is connected to a respective end of the connector so that each tube forms opposite ends of a spring clip.

Preferably the LED is surrounded by a substantially bullet-nosed tip 19 adapted to emit the NIR/red light. In some embodiments thereof, the bullet-nosed tip is substantially transparent. In these embodiments, it is desirable for the distal portion of the emitter to further include a lens (not shown) that focuses the emitted light towards a more posterior direction. In other embodiments, a first portion of the bullet-nosed tip has a reflective interior surface 23 while a second portion 25 of the bullet-nosed tip is substantially transparent, thereby allowing the NIR/red light to exit the device in a direction that is more posterior than the longitudinal axis (LA) of the emitter.

In one embodiment, the tubular portions of each device are connected at each end portion of a springy curved connector 3 so that each tube forms opposite ends of a spring clip. In some embodiments, the connector is made of a shape memory material (such as Nitinol) having a memorized shape that forms an angle alpha (as shown in FIG. 6C), and a loaded shape (FIG. 6A) that reflects the insertion of the tubes into the nostrils.

In some embodiments, the springy curved connector is substantially the only portion of the device that resides outside the nostril. In some embodiments, at least a portion of the springy curved connector is flesh-colored or clear. Preferred flesh colors include tan, brown and beige.

Preferably, each tube has a longitudinal axis and the connector provides a spring action so that, when unloaded, the longitudinal axes of the tubes form an angle α of at least 5 degrees, preferably at least 10 degrees, more preferably at least 20 degrees, and more preferably at least 30 degrees. In some embodiments, when the device is unloaded (FIG. 6B), the distal end portions of the emitters touch. In some embodiments, when the device is unloaded, the distal end portions of the emitters are separated by a distance of no more than 90% of the diameter of the tubes, preferably no more than 75%, more preferably no more than 50%, more preferably no more than 25%.

In some embodiments, the device has markings indicating a front and back of the device. This allows the user to guarantee insertion of the device into the nostril in an orientation that directs the red/NIR light towards a more posterior region of the skull. Preferably, these markings are on the spring clip connector. In one embodiment, the word “FRONT” is on the front of the clip. In other embodiments, the markings are on the tubes.

In some embodiments, the light emitters are actuated by simply turning the threaded knobs in a predetermined direction so as to advance the knob towards its respective tube. The distal advance of the knob activates a circuit and actuates the LED, thereby causing NIR/red light emission. To turn off the light emission, the knob is turned in the reverse direction.

In some embodiments, the light emitters are actuated by an on/off push-button feature located in the proximal portion of the device.

In some embodiments, the device incorporates electronics. In some embodiments, the electronics is adapted to cause a pulsing of the light. In others, the electronics is adapted to turn off the emission after a set time period of actuation. In some embodiments, the set time period of emission is between 10 and 30 minutes.

Further preferred features of the device and method of use can be found in U.S. Pat. No. 7,351,253, the specification of which is incorporated by reference in its entirety.

It is noted that commercially available balloon party lights have substantially the same dimensions as the human nostril. Therefore, in some embodiments, the design of one of these commercially available balloon party lights is adapted by simply replacing the LED source with an NIR LED.

In some embodiments, the light emitting from the device is preferentially directed off the longitudinal axis of the device as it leaves the device. In some embodiments, this is performed with a lens contained within the device. In other embodiments, this is performed by surrounding the LED unit with a reflective surface except for one substantially transparent surface, wherein the substantially transparent surface is oriented off the longitudinal axis of the device substantially in the direction of the olfactory bulb.

It is believed that three conventional wristwatch batteries, each carrying 1.5 volts, is sufficient to power an LED to emit an effective amount of red/NIR light. In one embodiment, the battery type is LR41 (1.5 volts). The sum of 4.5 volts is substantially equivalent to the 5 volts carried by the conventional 5 milliwatt laser pointer. It is believed that the present device should have a power of 5 milliwatts. In comparison, the Vielight® device is powered by about volts to produce a current of about 5-8 milliwatts. It is believed that using three such batteries will provide sufficient power to the device for a period of about 5-8 hours. Therefore, in some embodiments, the device is actuated for between about 10 and about 30 minutes each day for about a week, and then discarded at the end of the week.

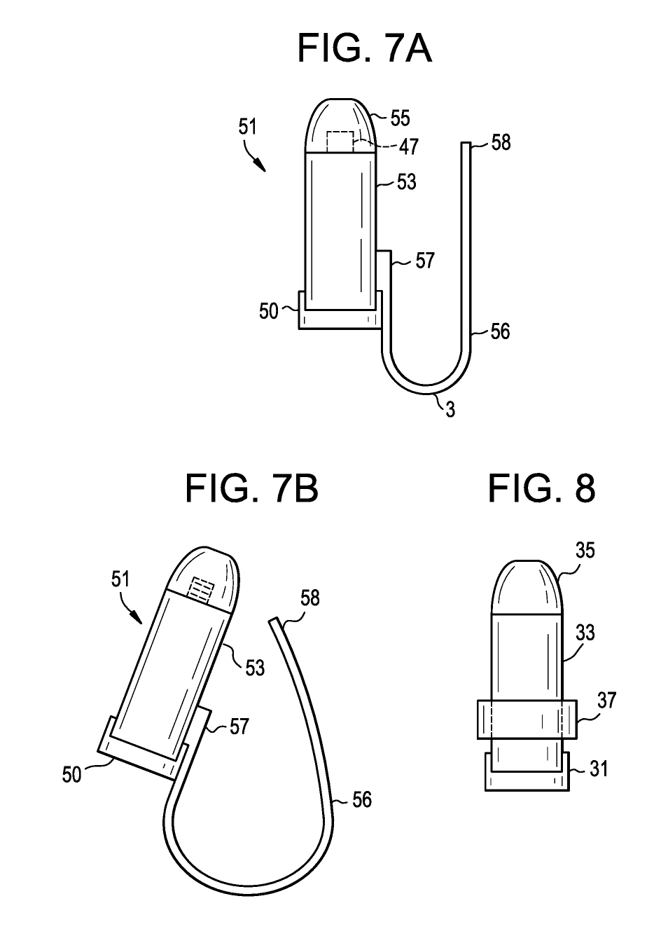

Now referring to FIGS. 7 A-B, in some embodiments, the device has a unilateral infrared/red light emitter. Preferably, in this embodiment, the device comprises:

a) an emitter 51 having:
i) a proximal portion comprising an LED actuator (such as a threaded on/off knob 50);
ii) an intermediate portion comprising a cylindrical tube 53 that houses batteries in its bore and a proximal mating thread, and
iii) a distal portion 55 comprising an LED 47, and
   b) a springy curved connector 56 having opposite first and second ends 57 and 58,
   wherein the tube of the emitter is connected to a first end 57 of the connector so that the tube and second end of the connector form opposite ends of a spring clip.

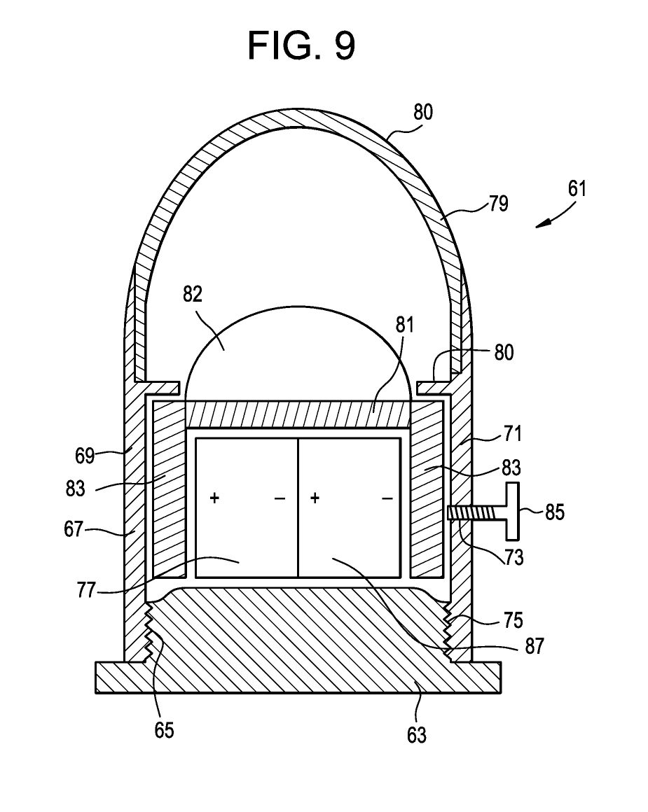

Now referring to FIG. 8, in other embodiments, the device comprises an emitter having:
i) a proximal portion comprising an LED actuator (such as threaded on/off knob 31);
ii) an intermediate portion comprising a substantially cylindrical tube 33 that house batteries in their bores and a proximal mating thread, and
iii) a distal portion 35 comprising an LED,
   wherein the outer surface of the tube is coated with an adhesive 37.

In some embodiments, a double sided adhesive tape is attached to the outer surface of the tube so that the protective layer faces outward. Preferably, the adhesive extends substantially peripherally about the tube. When the device is ready to be inserted, the protective layer is removed and the device in inserted into the nose. The dimensions of the tube and the adhesive layer are such that the device fits snuggly within the nostril and remains in place even under human activity.

In other embodiments, the intranasal device comprises an emitter having:
i) a proximal portion comprising an LED actuator (such as threaded on/off knob);
ii) an intermediate portion comprising a substantially cylindrical tube that house batteries in their bores and a proximal mating thread, and
iii) a distal portion comprising an LED,
   wherein the outer surface of the tube is covered with a resilient material (such as foam rubber or a shape memory material).

The resilient material has the property of narrowing under pressure as the device passes through the narrow outer rim of the nostril, and then widening once it is within the wider nostril proper. This resilient property provides the device with a ledge element that keeps the device within the nostril until such time the device needs to be removed.

In some embodiments, a portion of the device that resides outside the nostril has an ornamental element. The ornamental element is particularly advantageous in the unilateral embodiments in which a portion of the device resides laterally outside the nostril in which the device resides. In other embodiments of the bilateral nature, the ornamental element resides on the springy connector between the nostrils.

If the LED is an infrared LED, then in some embodiments, the device has a second LED in the distal portion emitting light in the visible range. This enables the user to know whether or not the device is turned on. Preferably, the visible LED emits red light.

In some embodiments, there is provided an intranasal light-emitting device comprising:
i) a proximal portion comprising a threaded cap comprising a near infrared LED actuator (preferably comprising a threaded on/off knob);
ii) an intermediate portion comprising a tube (preferably a substantially cylindrical tube) that houses a power source in its bore and a proximal mating thread, and
iii) a distal portion comprising a near infrared LED,
   wherein the dimensions of the device are such that the device fits snuggly within the nostril and remains in place even under human activity.
   Preferably the near infrared LED is surrounded by a substantially bullet-nosed tip that is distally pointing and adapted to emit the NIR light. In some embodiments thereof, at least a portion of the bullet-nosed tip is substantially transparent.

For the purpose of the present invention, a “near infrared LED” is a light emitting diode whose maximum emission occurs at a wavelength in the near infrared range.

In some embodiments, the length of the device is such that at least 70% of the device fits inside the patient's nose, preferably at least 80%, more preferably at least 90%, more preferably at least 95%.

In some embodiments, the length of the device is less than 5 times the diameter of the device, preferably less than 4 times the diameter of the device, and more preferably the length of the device is about 3 times the diameter of the device. When these dimensional requirements are met, the device can both snugly fit inside the nose and not protrude significantly from the nostril.

Preferably, the LED is in electrical connection with the power source (which is preferably at least one battery and more preferably a plurality of batteries).

In some embodiments, there is provided an intranasal light-emitting device comprising:
i) a proximal portion comprising a threaded cap comprising an LED actuator;
ii) an intermediate portion comprising a tube that houses a power source in its bore and a proximal mating thread, and
iii) a distal portion comprising a near infrared LED, wherein the length of the device is such that at least 70% of the device fits inside the patient's nose.
   In some embodiments, there is provided an intranasal light-emitting device comprising:
i) a proximal portion comprising a threaded cap comprising an LED actuator;
ii) an intermediate portion comprising a tube that houses a power source in its bore and a proximal mating thread, and
iii) a distal portion comprising a near infrared LED,
   wherein the near infrared LED is surrounded by a substantially bullet-nosed tip that is distally pointing and adapted to emit the NIR light.

Preferably, at least a portion of the bullet-nosed tip is substantially transparent.

Preferably, the tip comprises a lens.

Preferably, a first portion of the bullet-nosed tip has a reflective interior surface and a second portion of the bullet-nosed tip is substantially transparent.

In one embodiment, the NIR LED is the VSLY5850 (850 nm) LED, manufactured by Vishay in Malvern, Pa. or Shelton, Conn.

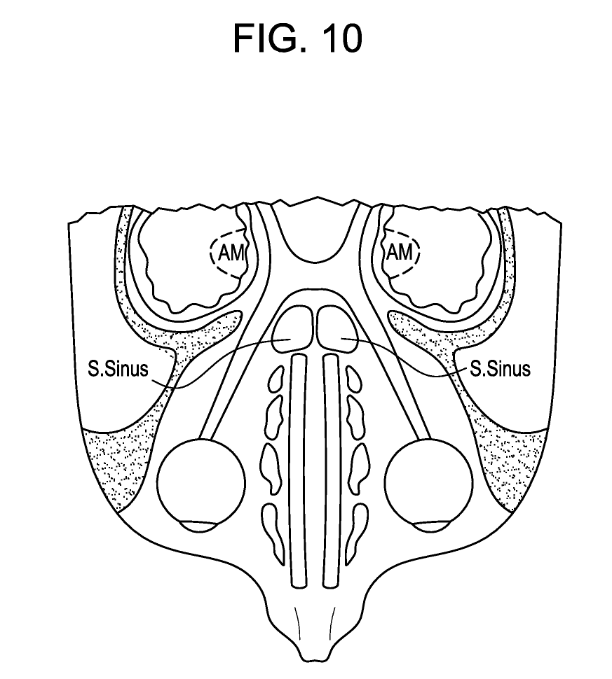

In some embodiments, and now referring to FIG. 9 there is provided an intranasal light-emitting device 61 comprising:
i) a proximal portion 63 comprising a connector 65;
ii) an intermediate portion 67 comprising a tube 69 having a longitudinal axis, wherein the tube has a sidewall 71 having a threaded hole 73 therethrough and a proximal mating connector 75,
iii) a power source 77 housed within the bore of the tube,
iv) a distal portion 79 comprising a transparent dome 80, the distal portion attached to the intermediate portion,
v) a near infrared LED 81 housed within the tube and having first and second leads 83,
vi) a screw 85 threadably extending through the threaded hole in the tube,
   wherein the leads terminate at substantially the same longitudinal level, and
   wherein the power source comprises at least one battery 87 having first and second opposite poles wherein the poles form a second axis that is substantially perpendicular to the longitudinal axis,
   wherein the second pole contacts the second lead,
   wherein the screw has a retracted position in which the screw does not contact the first lead and an extended position in which the screw biases the first lead to contact the first pole, thereby provided a circuit.
   Preferably, the LED is encased in a casing 82.
   Preferably, the tube comprises flanges 80 adapted to keep the LED centered within the tube.
   Preferably, the dome is sized so as to be friction fit within the tube.

Moreover, since red light experiences high diffraction as it proceeds through soft tissue, it is possible for the entire lower portion of the prefrontal cortex to be irradiated with red light.

Therefore, in accordance with the present invention, there is provided a method comprising the steps of:
a) providing an device having a NIR/red light emitter,
b) positioning the emitter within a nasal cavity, and
c) activating the light source to irradiate brain tissue with an effective amount of NIR/red light.

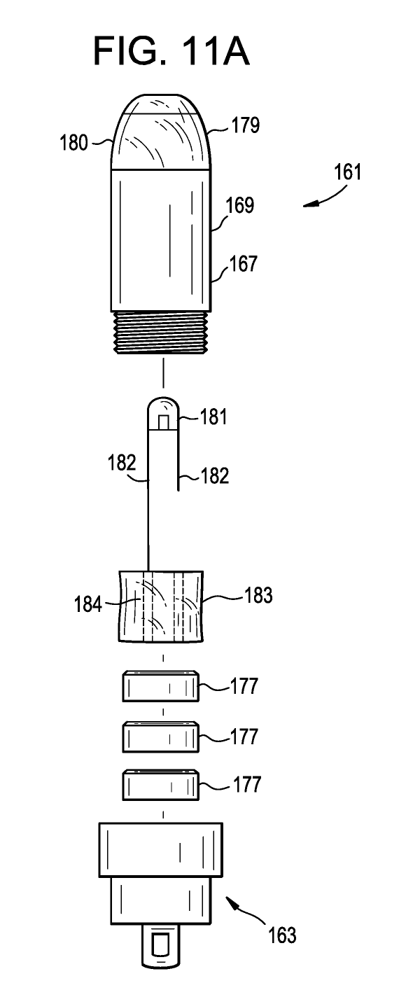

Studies on human cadaveric heads have revealed that intranasal delivery of red or infrared light irradiates not only the anteriomedial portion of the orbitofrontal cortex, but the posteromedial portion of the orbitofrontal cortex as well. Thus, with wishing to be tied to a theory, it is believed that there exists an anterior-posterior corridor in the upper nasal cavity that is amenable to irradiating entire medial orbitofrontal cortex because that corridor is substantially air. FIG. 10 is an axial cross-section of the head at the level of the upper nasal cavity and it shows the presence of many pockets within the nasal cavity. These pockets help transport light with little attenuation. In contrast, it is believed the lower nasal cavity has much more tissue and so does not diffuse light as well, but rather significantly attenuates it. Thus, in the upper nasal cavity, for at least a portion of the test subjects, the intensity of light in the anterior portion of the upper nasal cavity was substantially equal with that in the corresponding posterior portion.

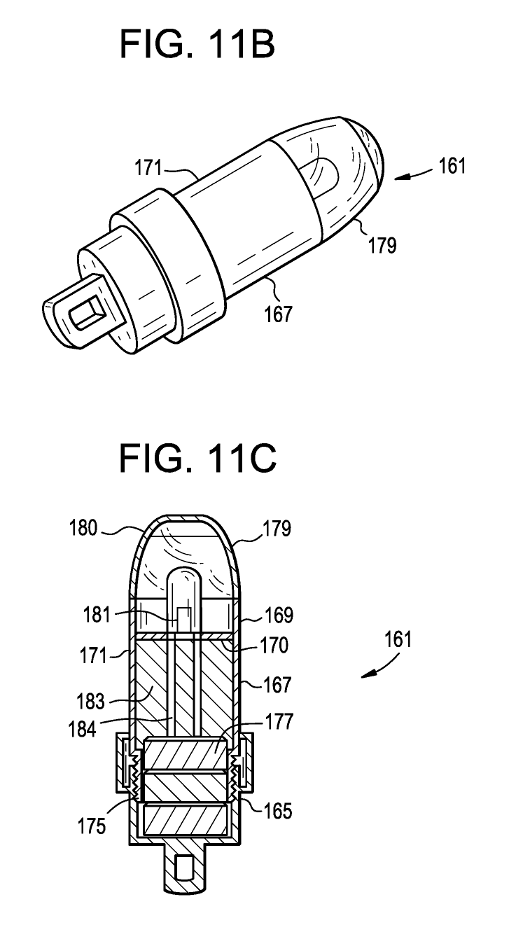

In some embodiments, and now referring to FIG. 11A-C there is provided an exploded, perspective and cross-sectional versions of an intranasal light-emitting device 161 comprising:
i) a proximal portion 163 comprising a connector 165;
ii) an intermediate portion 167 comprising a tube 169 having a longitudinal axis, wherein the tube has a sidewall 171 having an inwardly extending ledge 170 and a proximal mating connector 175,
iii) a power source comprising three batteries 177 housed within the bore of the tube, iv) a distal portion 179 comprising a transparent dome 180, the distal portion attached to the intermediate portion,
v) a near infrared LED 181 housed within the tube and having first and second leads 182, wherein a first lead contacts the proximal pole of the proximal battery and a second lead contacts the distal pole of the distal battery, and
vi) a cylindrical separator 183 disposed between the LED and the power source, the separator having two axial throughholes 184 through which the respective leads pass.

In some embodiments, an insulating sleeve (not shown) surrounds the batteries.

### Theory

Without wishing to be tied to a theory it is believed that red or infrared light induces synaptic plasticity in the areas of the brain that are under distress, disordered or damaged, but leaves normal regions unaffected. This enhanced plasticity leads not only to a beneficial local increase in the cortical volume, but also to a beneficial functional reorganization in the network of the affected area.

There have been numerous studies reporting the beneficial effect of transcranial NIR light upon depressed patients: Schiffer, Behav. Brain Funct., 2009 Dec. 8; 5:46 reported the psychological benefits 2 and 4 weeks after a single treatment with near infrared light to the forehead in a pilot study of 10 patients with major depression and anxiety. P. Cassano et al., Psychiatry J. 2015, 352979 (2015) reported on a proof of concept study for near-infrared transcranial radiation for major depressive disorder in which 2 of the 4 treatment completers demonstrated remission. M. A. Naeser et al., J. Neurotrauma 31(11), 1008-1017 (2014) reported significant improvements in cognitive performance after transcranial and intranasal red/near-infrared light-emitting diode treatments in chronic, mild traumatic brain injury, in which three subjects (37%) showed antidepressant response (decrease of BDI-II total score ≥50% from baseline) after 6 weeks of PBM treatment (mean BDI-II total scores were 14.8 6.5 SD). Two of them maintained the antidepressant response after 8 additional weeks of follow-up.

There have been numerous studies implicating the postero-medial orbitofrontal cortex/gyrus rectus region in depression:
Bremner, Biol. Psychiatry, 2002 Feb. 15; 51(4):273-9 reported that patients with depression had a statistically significant 32% smaller medial orbitofrontal (gyms rectus) cortical volume, without smaller volumes of other frontal regions including anterior cingulate Brodmann's area 24 (subgenual gyms), anterior cingulate Brodmann's area 32, subcallosal gyms (Brodmann's area 25), or whole brain volume. The findings were significant after statistically controlling for brain size. Ballmaier, Am J Psychiatry 2004; 161:99-108 concluded that prominent bilateral gray matter deficits in the anterior cingulate and the gyms rectus (20-24%) as well as the orbitofrontal cortex may reflect disease-specific modifications of elderly depression.
Grieve, Neuroimage Clinical 3 (2013) 332-339 reported widespread reductions in gray matter volume in depression, including that cluster maxima corresponding to the medial OFC were present in the middle frontal gyms (BA 10/11) and the gyms rectus. The decreased GM volume in this cluster corresponded to a difference of approximately 8-10% (CVNA: 23-25 years). The changes were anatomically symmetric. The gyms rectus GM differences (26%) were profound but restricted to the posterior portion (CVNA: N50 years). For the lateral OFC, the peak cluster was located in the middle frontal gyms (Gm difference of 19%, CVNA: 24 years).
Accolla, J. Affect Disorder, 2016 April, 194, 33-7 observed in one patient an excellent clinical response after deep brain stimulation (DBS) of the bilateral posterior gyms rectus rather than the initially targeted CG-25. The remaining four patients with DBS of the CG-25 were considered as non-responders. The case patient demonstrated a strong connectivity of the stimulated regions to the medial prefrontal cortex (mPFC), which contrasted to the lower mPFC connectivity in non-responders.
As for bipolar disorder, Almeida, Psychiatry Research., 2009 Jan. 30, 171(1) 54-68 reported that Bipolar type I patients showed reduced grey matter volume in only two regions established a priori: each of the bilateral posteromedial gyms rectus regions.
   Because these papers demonstrate that neural correlates of depression and bipolar disorder lie in the regions implicated by the red zone produced by trans-oral therapy, it is believed that intranasal red/NIR therapy will have a beneficial effect on bipolar or depressed patients.

Similarly, the neural correlates of anxiety appear to reside in the orbitofrontal cortex in the region that lies above the anterior-posterior nasal corridor described above:
Both the gyms rectus and amygdala are implicated in anxiety. Sladky, PLOSOne, November 2012, 7, 11, e50050
Structural correlates of trait anxiety as a reduced thickness in the medial orbitofrontal cortex. Kuhn, Journal of Affective Disorders, Volume 134, Issues 1-3, November 2011, Pages 315-319
OFC thickness mediates anxiety. Blackmon, Psychiatry Res. 2011 December, 194, 3, 296-303.
OFC thickness mediates optimism, which mediates anxiety, and suggests growing the OFC as a protective measure against anxiety. Dolcos, Social Cognitive and Affective Neuroscience, 2016, 263-271;
Advances in neuroimaging techniques over the past two decades have allowed scientists to investigate the neurocircuitry of anxiety disorders. Such research has implicated the orbitofrontal cortex (OFC). Characterizing the role of OFC in anxiety disorders, however, is principally complicated by two factors-differences in underlying pathophysiology across the anxiety disorders and heterogeneity in function across different OFC sub-territories. Contemporary neurocircuitry models of anxiety disorders have primarily focused on amygdalo-cortical interactions. The amygdala is implicated in generating fear responses, whereas cortical regions, specifically the medial OFC (mOFC) and the ventromedial prefrontal cortex (vmPFC), are implicated in fear extinction. Milad, Ann N Y Acad Sci. 2007 December; 1121:546-61. Epub 2007 Aug. 14.

### Red Light Biology

It has been reported in the literature that red/near infra-red light saves neurons that have been challenged by neurotoxics from apoptosis. In particular, Wong-Riley, J Biol Chem. 2005 Feb. 11; 280(6):4761-71. reports that irradiating neurons with 670 nm red light significantly reduced neuronal cell death induced by 300 mM KCN from 83.6% to 43.5%.

The general concept of repairing brain cells through red/NIR light irradiation is also well supported by the literature. Wollman, Neurol. Res. 1998, Jul. 20(5) 470-2 reports that providing daily 3.6 J/cm² doses of red light from a He—Ne laser to cortex explants resulting in caused a significant amount of sprouting of cellular processes outgrowth. Wollman concludes that the irradiation induces neurite processes sprouting and improves nerve tissue recovery. Similarly, Wollman, Neurol. Res. 1996 Oct. 18(5) 467-70 reports the enhanced migration and massive neurite sprouting of cultured rat embryonal brain cells subject to an 8 minute dose of a 0.3 mW, He—Ne laser. Therefore, the red/NIR light of the present invention may further cause repair and regeneration of damaged neuronal cells.

Without wishing to be tied to a theory, it is believed that the therapeutic neuroprotective and neuroregenerative effects of red/NIR light described above may also be due to a) an increase in ATP production in the irradiated neurons, and b) an increase in the activity of local anti-oxidant enzymes superoxide dismutase (SOD) and catalase.

It is believed that irradiating neurons in the brain with red light will likely increase ATP production from those neurons. Mochizuki-Oda, Neurosci. Lett. 323 (2002) 208-210, examined the effect of infrared light on energy metabolism in the rat brain and found that irradiating neurons with 4.8 W/cm² of 830 nm infrared light increased ATP production in those neurons by about 19%.

Without wishing to be tied to a theory, it is further believed that the irradiation-induced increase in ATP production in neuronal cell may be due to an upregulation of cytochrome oxidase activity in those cells. Cytochrome oxidase (also known as complex IV) is a major photoacceptor in the human brain. According to Wong-Riley, Neuroreport, 12:3033-3037, 2001, in vivo, light close to and in the near-infrared range is primarily absorbed by only two compounds in the mammalian brain, cytochrome oxidase and hemoglobin. Cytochrome oxidase is an important energy-generating enzyme critical for the proper functioning of neurons. The level of energy metabolism in neurons is closely coupled to their functional ability, and cytochrome oxidase has proven to be a sensitive and reliable marker of neuronal activity.

By increasing the energetic activity of cytochrome oxidase, the energy level associated with neuronal metabolism may be beneficially increased. Indeed, the literature reports that NIR/red light reverses the inhibitory effects of neurotoxins upon cytochrome oxidase activity, leading to increased energy metabolism in neurons functionally inactivated by toxins. Wong-Riley Neuroreport 12(14) 2001:3033-3037 and Wong-Riley, J. Biol. Chem, supra.

According to Kamanli, Cell Biochem. Func. 2004, 22:53-57, catalase detoxifies hydrogen peroxide and converts lipid hydroperoxides into non-toxic alcohols, and is essential for the inhibition of inflammation related to the function of neutrophils. Romm, Biull. Eksp. Biol. Med. 1986 October 102(10) 426-8 reports that laser irradiation of wounds results in a decreased chemiluminescence that is attributable to activation of catalase in the tissue fluid.

Therefore, it is believed that irradiating an affected brain with an effective amount of NIR/red light will therapeutically increase of the activity of catalase in the irradiated region, thereby attenuating the deleterious effect of hydrogen peroxide upon the neurons in the affected brain. According to Kamanli, supra, SOD catalyses dismutation of the superoxide anion into hydrogen peroxide. The literature repeatedly reports that red/NIR light irradiation of inactivated SOD increases its activity. For example, Vladimirov, Biochemistry (Moscow) 69(1) 2004, 81-90 provides a review including the photoreactivation of Cu—Zn SOD under He—Ne laser. Karu, Laser Ther. 1993, 5, 103-9 reports that reactive oxygen species in human blood were found to be suppressed after laser diode illumination at 660 nm, 820 nm, 880 nm and 950 nm. This affect has been attributed by other authors to the activation of SOD or catalase. Volotovskaia Vopr Kurortol Zizioter Lech Fiz Kult 2003 May-June (3)22-5 reports that 632 nm He—Ne laser irradiation of blood has an anti-oxidant effect as shown by activation of SOD. Ostrakhovich Vestn Ross Akad Med Nauk. 2001(5) 23-7 reports that infrared pulse laser therapy of RA patients caused an increase in SOD activity. Gorbatenkova Biofizika, 1988 July-August 33(4) 717-9 reports that SOD that was inactivated by hydrogen peroxide was reactivated by a 450-680 nm red light laser. Vladimirov, Free Rad. Biol. Med. 1988, 5(5-6) 281-6 reports the inactivation of SOD by its incubation in a low pH 5.9 solution and its subsequent reactivation by helium-neon laser light. Catalase was found to be reactivated as well. Cho, In Vivo, 2004, September-October 18(5) 585-91 reports on the use of low level laser therapy (LLLT) to treat knee joints that have been induced with OA by injection of hydrogen peroxide. SOD was reported to increase about 40% in the OA group as compared to controls.

Therefore, it is believed that irradiating the affected brain with an effective amount of red/NIR light will therapeutically increase of the activity of SOD in the irradiated region, thereby attenuating the deleterious effect of superoxide anion upon the neurons in the distressed brain.

According to Leung, Laser Surg. Med. 31:283-288 (2002), nitric oxide enhances oxidative insult by reacting with superoxide anion to form a stronger oxidant, peroxynitrite, which leads to mitochondrial dysfunction, DNA damage and apoptosis. Leung, supra, investigated the effect of low energy red laser after stroke in rats, and found that red light can suppress NO synthase activity. In particular, Leung found that irradiating a portion of the rat's brain with a 660 nm red light (average power 8.8 mW, 2.64 J/cm²) reduced NOS activity up to about 80% over that in unirradiated stroke rats, and up to about 60% over the NOS activity in normal rats. Leung concluded that low energy laser may be protective by suppressing the activity of NOS.

Without wishing to be theory, it is believed that irradiation of a portion of a distressed brain will similarly therapeutically suppress NO synthase activity, thereby attenuating peroxynitrite activity.

It is noted that Leung, supra, also reported that red/NIR light irradiation of the brain resulted in a TGF-β tissue concentration of 1-6 ng/ug protein of tissue. Thus, red light irradiation of the OFC may very well be an attractive non-invasive way of generating large amounts of TGF-β within the brain.

Moreover, the literature has reported other highly beneficial effects of red light, including its attenuation of the immune response following neuronal injury. Byrnes, Lasers Surg. Medicine 9999:1-15(2005) reports that 810 nm light promotes the regeneration and functional recovery of the injured spinal cord, and significantly suppressed IL-6 and iNOS expression and immune cell activation. Of note, Byrnes reports a 171-fold decrease in IL-6 expression and an 80% reduction in iNOS expression when the spinal cord lesion was irradiated on a daily basis with about 100 J/cm² red light for about 2 weeks.

Therefore, in light of the above studies, and without wishing to be tied to a theory, it is believed that the decreased cortical volume and disorder of the medial OFC and gyms rectus play a predominant and direct role in the depressed state of the PND mother. It is further believed that these deficits can be reversed by low level laser therapy (“LLLT”) treatment of these regions with red/near infrared light (“red/NIR light”). In particular, it is believed that red/NIR light will beneficially act upon the medial OFC and gyms rectus through the following avenues:

a) increasing the amount of ATP;

b) increasing the amount of BDNF;

c) increasing the amount of bcl-2, and

d) increasing the amount of sprouting, leading to network reorganization.

Oron, Photomed Laser Surg. 2007 June; 25(3):180-2. (2007) reports that in vitro red/NIR light approximately doubles the amount of ATP in neurons. Since metabolic processes of the brain substantially use ATP as their fuel, it is believed that the increase in ATP afforded by LLLT will help normalize the OFC and gyms rectus.

Zhang Cell Physiol Biochem. 2008; 22(1-4):215-22 reports that LLLT activates PKC in neurons within one hour of the irradiation (Zhang, 2008). Because it is known that PKC increases serotonin release from synapse (Psychopharmacology (Berl). 1989; 99(2): 213-8.) and PKC has been implicated in mediating neuronal plasticity, and that increasing the availability of serotonin provides a positive benefit for depressed patients, the activation of PKC should provide a therapeutic benefit to the depressed patient.

As discussed above, it is now believed that the effectiveness of many conventional antidepression treatments may lie in their ability to induce neurotrophins such as brain-derived neurotrophic factor (BDNF), in the patient's brain. It has been shown that LLLT acts upon neurons to increase BDNF 5× in neurons (Byrnes Lasers Surg Med. 2005 August; 37(2):161-71.), and (Anders IEEE J. Quantum Electronics, 14/1 January/February 2008, 118-125). The 5×BDNF induction produced by LLLT compares favorably with the increase in BDNF induced by antidepressants, and is approximately the same level of BDNF induction generated by ECT.

bcl-2 is an anti-apoptotic gene that has been implicated in mediating neuronal plasticity. Manji, Psychopharmacol Bull. 2001 Spring; 35(2):5-49 and Manji, Am J Psychiatry. 2005 April; 162(4):805-7 report that bcl-2 expression correlates with clinical benefit of antidepressants. In this respect, red light has been shown to increase bcl-2 in neurons (Liang, Neuroscience. 2006 May 12; 139(2):639-49,) and (Zhang, supra, 2008)

Further without wishing to be tied to a theory, it is further believed that red/NIR light therapy of the medial OFC and gyms rectus will provide a number of advantages to the patient.

First, red/NIR light therapy is a completely non-toxic therapy. Thus, it appears that its use poses no known danger to the patient. Therefore, red/NIR light therapy/LLLT can be used by a nursing mother without any apparent risk to the child.

Second, it is believed that red/NIR light therapy will work much more quickly than conventional antidepressants, with LLLT providing a first round of benefit within about an hour of the initial irradiation and a second round of benefit within a few days of the initial irradiation.

Respecting highly acute events, Oron (supra, 2007) reports that in vitro red/NIR light increases ATP in neurons within 10 minutes of the application of red/NIR exposure, while Zhang reports that LLLT activates PKC in neurons within one hour of the irradiation (Zhang, supra, 2008). Thus, two mechanisms are acting favorably upon the patient within an hour of LLLT treatment.

Respecting more subchronic events, Anders, 2008 reports that red/NIR light increases BDNF in neurons within 3-7 days of the beginning of red/NIR light exposure. Zhang (2008)/Liang & Whelan (2006) report that red/NIR light increases bcl-2 in neurons within 6-28 hours respectively of the beginning of red/NIR light exposure. In contrast, Wada 2005 reports that only chronic use of lithium upregulates bcl-2 expression (Wada, J Pharmacol Sci. 2005 December; 99(4):307-21.) and only chronic lithium correlates with clinical benefits (Wada 2005)

Further evidence of the quick acting nature of red light is found in studies examining the effect of red light upon neuronal sprouting. Wollman, Neurol. Res., 1998, 20: 470-2 reported that human cortical explants exposed to red/NIR light displayed significantly increased sprouting after only 6 days. Also, Rochkind, Lasers Surg. Med. 41, 277-281(2009) reported that 780 nm light irradiation of embryonic rat brain cultures induced rapid sprouting of nerve processes within 24 hours and a 3-fold increase in large perikaryae after 4 days. Rochkind reported that this “precocious appearance of large neurons is unlike the usual growth pattern in which neurons grow and become large only after several weeks in culture.” Rochkind found that irradiation at 50 mW for one minute induced the most significant change. Therefore, because of the red/NIR light irradiation-induced induction of early sprouting, it is believed that the NIR/red light device of the present invention may be able to provide a clinical benefit to the patient within a few days of beginning treatment.

The present inventors are aware of at least two reports of very favorable effects of red/NIR light irradiation of neuronal cells at fluences (doses) of less than 1 J/cm². As discussed above, Byrnes, Lasers Surg Med. 2005 August; 37(2):161-71 found a significant (P<0.05) increase in brain derived neurotrophic factor (BDNF) and glial derived neurotrophic factor (GDNF) in the 0.2 J/cm² group in comparison to the non-irradiated group. Oron, Photomed Laser Surg. 2007 June; 25(3):180-2 reports that normal human neural progenitor (NHNP) cells were grown in tissue culture and were treated by Ga—As laser (808 nm, 50 mW/cm², 0.05 J/cm²). They found that the quantity of ATP in laser-treated cells 10 minutes after laser application was 7513+/−970 units, which was significantly higher (p<0.05) than the non-treated cells, which comprised 3808+/−539 ATP units. In sum, Oron found that the neuronal ATP level was essentially doubled by infrared LLLT. In addition, Byrnes, Lasers Surgery Medicine, March 2005, 36(3) 171-85 reports that dosages as low as 0.001 J/cm² stimulate cellular activity (such as DNA, RNA and protein production, proliferation and motility). Therefore, it is believed that fluences as low as about 0.01 J/cm² (and possibly even about 0.001 J/cm²) will be effective in providing therapy to the pertinent neurons of the perinatal depression (PND) patient.

In some embodiments, the light source is situated to produce about 1-90 milliwatt/cm², and preferably 7-25 milliwatt/cm² of irradiation upon the cortical surface.

In accordance with US Patent Publication 2004-0215293 (Eells), LLLT suitable for the neuronal therapy of the present invention preferably has a wavelength between 630-1000 nm and power intensity between 25-50 mW/cm² and is provided for a time of 1-3 minutes (equivalent to an energy density of 2-10 J/cm²). Eells teaches that prior studies have suggested that biostimulation occurs at energy densities between 0.5 and 20 J/cm². Wong-Riley. J. Biol. Chem. 2005 Feb. 11, 280(6), 4761-71 reports that fluences as high as 30 J/cm² appear to be effective in preventing cell death in neurons exposed to the mitochondrial poison KCN. In some embodiments, the preferable energy density of the present invention is between 0.1 and about 30 J/cm², more preferably between 0.5-20 J/cm², most preferably between 2-10 J/cm². In summary, a preferred form of the present invention uses red and near infrared (red/NIR) wavelengths of 630-1000, most preferably, 670-900 nm (bandwidth of 25-35 nm) with an energy density fluence of 0.5-20 J/cm², most preferably 2-10 J/cm², to produce photobiomodulation. This is accomplished by applying a target dose of 1-90 mW/cm², preferably 25-50 mW/cm² LED-generated light for the time required to produce that energy density.

It is further believed that red/NIR light irradiation of neurons will produce a significant upregulation in brain derived neurotrophic factor (BDNF) and glial derived neurotrophic factor (GDNF). Byrnes, Lasers Surg Med. 2005 August; 37(2):161-71 reports that olfactory ensheathing OECs were purified from adult rat olfactory bulbs and exposed to 810 nm light (150 mW; 0, 0.2, or 68 J/cm²). Byrnes found that a significant (P<0.05) increase in BDNF, GDNF and collagen expression in the 0.2 J/cm² group in comparison to the non-irradiated and high dose groups.

Of note, it has been reported that the neuroprotective effects of red/NIR light can be effected by a single irradiation on the order of minutes. Wong-Riley, J. Biol. Chem. 2004, e-pub November 22, reports that irradiating neurons with 670 nm red light for only ten minutes results in neuroprotection. Similarly, Wong-Riley Neuroreport 12(14) 2001:3033-3037 reports that a mere 80 second dose of red light irradiation of neuron provided sustained levels of cytochrome oxidase activity in those neurons over a 24 hour period. Wong-Riley hypothesizes that this phenomenon occurs because “a cascade of events must have been initiated by the high initial absorption of light by the enzyme”.

In some embodiments, the red/NIR light irradiation is delivered in a continuous manner. In others, the red/NIR light irradiation is pulsed (usually between 1 and 10 Hz) in order to reduce the heat associated with the irradiation. Without wishing to be tied to a theory, it is believed that pulsed light may be more effective in achieving the vibratory oscillation of the catalase and SOD molecules.

### Wavelength

Preferably, the light of the present invention has a wavelength of between about 600 nm and about 1100 nm. In some embodiments, the wavelength of light is between 800 and 900 nm, more preferably between 800 nm and 860 nm. In this range, red/NIR light has not only a large penetration depth (thereby facilitating its transfer to the fiber optic and SN), but Wong-Riley reports that cytochrome oxidase activity is significantly increased at 830 nm, and Mochizuki-Oda reported increased ATP production via a 830 mn laser. In some embodiments, the wavelength of light is between 600 and 700 nm. In this range, Wong-Riley reports that cytochrome oxidase activity was significantly increased at 670 nm. Wollman reports neuroregenerative effects with a 632 nm He—Ne laser.

### Penetration Depth

Respecting penetration depths, Byrnes, Lasers Surg. Medicine 9999:1-15(2005) reports that an effective amount of 810 nm light was able to traverse a 1 cm thick rat spinal cord. The penetration depths of various wavelengths of red light in grey matter brain tissue have been reported in Yaroslavsky, Phys. Med. Biol. 47(2002) 2059-73 as follows:

| Wavelength | Penetration Depth (mm) |
|---|---|
| 630 nm | 0.83-4.06 |
| 675 nm | 1.29 |
| 670 nm | 4.4 |
| 1064 nm | 1.18-3.28 |

In general, the literature has reported that infrared light provided deeper penetration into brain tissue than red light. Tedford, Lasers in Surgery and Medicine 47:312-322 (2015). Therefore, in preferred embodiments, infrared light is used. Tedford further reports that infrared light attenuates in brain tissue at a rate of about 10 fold per centimeter.

### Fluence

In some embodiments, the light source is situated and applied to irradiate brain tissue with between about 0.02 J/cm² and 200 J/cm² energy. Without wishing to be tied to a theory, it is believed that light transmission in this energy range will be sufficient to increase the activity of the cytochrome oxidase and anti-oxidant activity around and in the neurons. In some embodiments, the light source is situated to irradiate target tissue with more than 10 J/cm², and preferably about 100 J/cm² energy. In some embodiments, the light source is situated to irradiate adjacent tissue with between about 0.2 J/cm² and 50 J/cm² energy, more preferably between about 1 J/cm² and 10 J/cm² energy.

### Intensity

In some embodiments, the light source is situated to produce an energy intensity of between 0.1 watts/cm² and 10 watts/cm². In some embodiments, the light source is situated to produce about 1 milliwatt/cm².

### Power

In some embodiments, the infrared LED irradiates with a power of at least 0.5 watts, more preferably at least 0.7 watts, more preferably at least 1 watt. In some embodiments, the infrared LED has a power of at least 3 watts, more preferably at least about 5 watts. In some embodiments, this high power LED is the SLTMAKS 10PCs/lot High power LED Chip 740 nm 850 nm IR LED 3 W 5 W Emitter Light Lamp LED Beads for LED Grow Light at aliexpress. Another high power 3 W 850 nm LED is available from Ledguhon at aliexpress. Another is the 10 Pcs Hontiey 3 W 850 nm LED Bo6XQDWQZT at amazon.

### Target Structures

It is believed that brain structures that can be beneficially irradiated with the device of the present invention include a) the medial OFC, and b) the gyms rectus. These structures have been implicated as neural correlates of depression.

### Timing

Of note, it has been reported that the neuroprotective effects of red light can be effected by a single irradiation on the order of minutes. Wong-Riley, J. Biol. Chem. 2004, supra, reports that irradiating neurons with 670 nm red light for only ten minutes results in neuroprotection. Similarly, Wong-Riley Neuroreport 12(14) 2001:3033-3037 reports that a mere 80 second dose of red light irradiation of neuron provided sustained levels of cytochrome oxidase activity in those neurons over a 24 hour period. Wong-Riley hypothesizes that this phenomenon occurs because “a cascade of events must have been initiated by the high initial absorption of light by the enzyme”.

Therefore, in some embodiments of the present invention, the therapeutic dose of red light is provided on approximately a daily basis, preferably no more than 3 times a day, more preferably no more than twice a day, more preferably once a day.

### Continuous/Pulsed

In some embodiments, the red/NIR light irradiation is delivered in a continuous manner. In others, the red/NIR light irradiation is pulsed in order to reduce the heat associated with the irradiation. Without wishing to be tied to a theory, it is believed that pulsed light may be more effective in achieving the vibratory oscillation of the catalase and SOD molecules.

### Applications

It is believed that the above devices are useful in treating brain disorders involving the orbitofrontal cortex, including traumatic brain injury, chronic traumatic encephalopathy, concussion, Alzheimer's Disease, depression, postpartum depression, hydrocephalus, frontotemporal dementia, and stroke involving the anterior cerebral artery.

### Lithium

As lithium's beneficial action upon patients with bipolar disorder and schizophrenia is also thought to be due to its ability to enhance neuroplasticity and reorganization, it is believed that the dose of lithium (which has certain unwanted side effects) given to these patients can be reduced by concomitant administration of lithium and red/infrared light. Therefore, there is provided a method of treating a patient with a brain disorder (preferably bipolar disorder or schizophrenia) comprising the steps of:

a) administering an effective amount of lithium to the patient, and

b) irradiating the patient's brain with an effective amount of infrared or red light,

wherein the effective amount of lithium administered is less than the dose required to treat the patient in the absence of red/infrared light administration.

### Behavior

In addition, simultaneous use of red/NIR light therapy with established behavior change interventions such as meditation, exposure therapy, cognitive behavioral therapy, and guided imagery can enhance the efficacy of those treatments.

A related approach for enhancing adherence purely for the stimulation of the brain is to use the Premack Principle. The Premack Principle states: If behavior B is of higher probability than behavior A, then behavior A can be made more probable by making behavior B contingent upon it. This is also known as “relativity theory of reinforcement”, based on the work of David Premack. As one example, if a person routinely drinks a cup of coffee, we would make that behavior incumbent on first using the red light therapy. Behaviors can also be simultaneously paired together in some cases e.g., use the device while reading the morning paper (e.g., “If you don't use the device, you don't get to read the paper”).

It is contemplated to use red/NIR light in conjunction with exposure therapy for fears, phobias and traumas. There are three ways to expose clients to their fears during systematic desensitization. First, exposure to fears can be accomplished through mental imagery. This approach can be more convenient and allows patients to complete treatment without ever leaving their therapist's office. Second, in vivo (direct exposure to the feared stimulus) is also possible. This option can be more complex (e.g., going to a dental office to provide exposure for a patient with a dental phobia), but appears to produce outcomes superior to imaginal exposure. Third, computer simulation (virtual reality) has been successfully used as a means of exposing a patient to feared stimuli. Simultaneous use of red/NIR light during these exposures might enhance the efficacy/benefit of the treatment.

### Uses

Professional use of these devices could include:
a) First Responders EMS—This could be part of every head injury assessment and treatment could begin immediately while the patient is being transported to the trauma center;
b) Military Battlefield—Much like the role of the EMS, first responders, this could be part of a field medic, special forces units, MASH units, and larger base hospitals, and
c) Sports Trainers, Coaches—Administration could begin immediately on the field after players sustain a concussion. This could be therapeutic and preventative for Chronic Traumatic Encephalopathy.

Consumer and self-administered uses of these devices could include:
a) Post-Partum Depression—Newly discharged mothers would use this in the peri-partum period, provided it was attractively packaged and marketed;
b) Antenatal depression—
c) Alzheimer's Disease|Benign Senile Forgetfulness—Senior citizens could administer treatments themselves or have in home care givers provide assistance for therapy. Venues would include assisted living and skilled nursing facilities;
d) Post Concussive Syndrome—Children and adolescents would benefit from therapy as many struggle with executive function tasks (math and reading) as they recover from sports related concussions. This has vast society implications;
e) First Aid Kits—Devices could be part of every First Aid kit for car, traveling, camping, home use, etc;
f) Acute Sports Injury|Concussion—Apart from the physical “contact” sports such as football, there potentially is a role for all sports including hiking, diving, particularly outdoorsman that are remote and not near medical care.
g) Obesity (wherein the gyms rectus is beneficially irradiated),
h) Major depression
i) Traumatic Brain Injury
j) Obsessive-Compulsive Disorder
k) Anxiety, and
l) CTE.

We claim:

1. An intranasal light-emitting device comprising:
i) a proximal portion comprising a connecting cap comprising an LED actuator;
ii) an intermediate portion comprising a tube that houses a power source in its bore and a proximal mating connector, and
iii) a distal portion comprising a near infrared LED,
wherein the dimensions of the device are such that the device fits snuggly within the nostril and remains in place even under human activity.

2. The device of claim 1 wherein the actuator comprises a threaded on/off knob.

3. The device of claim 1 wherein the power source comprises at least one battery.

4. The device of claim 1 wherein the near infrared LED is surrounded by a substantially bullet-nosed tip that is distally pointing and adapted to emit the NIR light.

5. The device of claim 4 wherein at least a portion of the bullet-nosed tip is substantially transparent.

6. The device of claim 1 wherein the length of the device is such that at least 70% of the device fits inside the patient's nose.

7. The device of claim 1 wherein the length of the device is about 3 times the diameter of the device.

8. The device of claim 1 wherein the the near infrared LED is surrounded by a hollow tip.

9. The device of claim 1 wherein the connecting cap and proximal mating connector comprise mating threads.

10. The device of claim 1 wherein the LED is in electrical connection with the power source.

11. A method comprising:
a) inserting into the patient's nostril a light-emitting device so that at least 70% of the device fits inside the patient's nostril, wherein the device comprises:
i) a proximal portion comprising a connecting cap comprising an LED actuator;
ii) an intermediate portion comprising a tube that houses a power source in its bore and a proximal mating connector, and
iii) a distal portion comprising a near infrared LED, and
b) actuating the device by placing the LED in electrical connection with the power source.

12. The method of claim 11 wherein at least 80% of the device fits inside the patient's nose.

13. The method of claim 11 wherein at least 90% of the device fits inside the patient's nose.

14. The method of claim 11 wherein the connecting cap and proximal mating connector comprise mating threads.

15. An intranasal light-emitting device comprising:
i) a proximal portion comprising a connecting cap comprising an LED actuator;
ii) an intermediate portion comprising a tube that houses a power source in its bore and a proximal mating connector, and
iii) a distal portion comprising a near infrared LED,
wherein the device further includes electronics adapted to provide a substantially constant current to the LED.

16. The device of claim 15 wherein the electronics is further adapted to provide a pulsed current to the LED.

17. The device of claim 15 wherein the electronics further includes a timer.

18. The device of claim 15 wherein the electronics is adapted to shut off current to the LED after a prescribed time-period.

19. The device of claim 15 wherein the electronics is adapted to alert the user that the power source is low.

20. An intranasal light-emitting device comprising:
i) a proximal portion comprising a connecting cap comprising an LED actuator;
ii) an intermediate portion comprising a tube that houses a power source in its bore and a proximal mating connector, and
iii) a distal portion comprising a near infrared LED,
wherein the near infrared LED is surrounded by a distal tip that is adapted to emit the NIR light distally.

21. The device of claim 20 wherein the power source comprises at least one battery.

22. The device of claim 20 wherein the distal tip is substantially distally pointing.

23. The device of claim 20 wherein the distal tip comprises a lens.

24. The device of claim 20 wherein a first portion of the tip has a reflective surface and a second portion of the tip is substantially transparent.

25. The device of claim 20 wherein the tip is substantially bullet-nosed.

26. The device of claim 20 wherein at least a distal portion of the distal tip is substantially transparent.

27. The device of claim 20 having substantially a single longitudinal dimension.

28. The device of claim 28 wherein the connecting cap and proximal mating connector comprise mating threads.

29. The device of claim 20 wherein the LED and power source are in electrical connection.
